(19) United States
(12) Patent Application Publication
(10) Pub. No.: US 2011/0071491 A1
(43) Pub. Date: Mar. 24, 2011

(54) Current Concentration System and Method for Electrokinetic Delivery of Medicaments

(75) Inventors: Michael S. Barsness, Oxford, MA (US); Robert W. Etheredge, Natick, MA (US); Dennis I. Goldberg, Sudbury, MA (US); Bireswar Chakraborty, Andover, MA (US); Shawn P. Davis, Boston, MA (US)

(73) Assignee: NITRIC BIOTHERAPEUTICS, INC.

(21) Appl. No.: 12/958,061

(22) Filed: Dec. 1, 2010

Related U.S. Application Data
(62) Division of application No. 12/139,936, filed on Jun. 16, 2008.
(60) Provisional application No. 60/944,126, filed on Jun. 15, 2007.

(51) Int. Cl. A61N 1/30

(52) U.S. Cl. 604/501

(57) ABSTRACT

An electrokinetic apparatus to apply medicament to a treatment site of a mammalian user, the apparatus including: a flux concentrator including an active electrode and a counter electrode adapted to be applied to the surface of a toenail or fingernail of the user, and a medicament matrix adjacent the active electrode and arranged to be sandwiched between the active electrode and nail.

### RELATED APPLICATION

The benefit is claimed of U.S. Patent Provisional Application Ser. No. 60/944,126, filed on Jun. 15, 2007, the entirety of which is incorporated by reference.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site on or under a toenail or fingernail or area of hard skin.

Electrokinetic delivery applies medication locally through the skin or nail of an individual to a treatment site typically in the skin or nail. One type of electrokinetic delivery mechanism is iontophoresis, i.e., the application of an electric field to the skin to enhance the skin's permeability and to deliver various ionic agents, e.g., ions of salts or other drugs to the treatment site. Iontophoretic or transdermal or transmucosal cutaneous delivery techniques have obviated the need for hypodermic injection of many medicaments thereby eliminating the concomitant problem of trauma, pain and risk of infection to the individual. Other types of electrokinetic delivery mechanisms include electroosmosis, electroporation, and electromigration, any or all of which are more generally known as electrotransport, electromolecular transport or iontophoretic methods, all of which are collectively known as electrokinetic methods.

Electrokinetic devices have been developed for the private self administration of medicaments or for diagnostic application by the individual at non-medical or non-professional facilities. For example, U.S. Pat. No. 6,792,306 and US Published Patent Applications 2006/0167403 and 2008/0051692, disclose electrokinetic delivery devices which include a housing containing a power source, electronics and a counter electrode, the device being shaped and configured for releasable secured to a finger and terminating in an applicator head having an active electrode. By applying the applicator head to the skin overlying the treatment site and with the medicament or a medicament and a carrier therefore carried by the applicator head, the medicament may be electrokinetically delivered to the treatment site. Similarly, US Published Patent Application 2006/0052737 discloses at FIGS. 25 and 26 electrokinetic devices for delivering medicament to a fingernail or toenail.

Electrokinetic devices may also be applied to delivery medicament to a treatment site below in or a toenail or a fingernail of a user. Medicament from the device flows into pores of the nail (or optionally through the pores) to the treatment site. The treatment site may be within the nail or in the skin tissue immediately below the nail. An electrical current flows from the device, the applicator sheet and into the nail to the treatment site. The electrical current promotes the flow of medicament through the nail (or to an upper region of the nail) and to the treatment site.

The electrical resistance of a nail, e.g., toenail or fingernail, is typically substantially greater than the soft tissue surrounding and below the nail. The current density applied by the electrokinetic device to the nail may be sufficiently high to cause skin irritation if current at the same density is applied to the soft skin tissue surrounding the nail. There is a risk that the application of high current by the device to apply medicament through a toenail or fingernail may inadvertently irritate the soft tissue surrounding the nail due to the current being unintentionally applied to the soft tissue.

There is a long felt need for an electrokinetic device and method that delivers medicament through a toenail or fingernail to a treatment site. Similarly, there is a long felt need for an electrokinetic device to deliver medicament into or through high resistance, e.g., toenails, or harden regions of the skin, such as callouses and warts. In particular, there is a need for an electrokinetic device capable of delivering medicament using a high current density applied to a toenail or fingernail (or high resistance or harden location on the skin) without high density current being applied to soft tissue near the treatment site.

### SUMMARY OF INVENTION

Systems and methods have been developed for focusing the electrical current applied, from an electrokinetic device to a fingernail or toenail (or other high resistance regions of the mammalian body such as harden skin regions) to deliver medicament through the nail and to a treatment site in or below the nail. The focused high density current from the device is directed at the toenail or fingernail, and the current density lessens as the current flows to soft tissue that may be under or adjacent the nail.

To focus the current, the device includes a flux concentrator that concentrates the current flow to a central region of the toenail or fingernail, and diffuses the return current flowing from the nail towards a counter electrode. The flux concentrator may include an active electrode and a surrounding counter (passive) electrode. The flux concentrator is positioned adjacent a medicament matrix that is applied to the toenail or fingernail. An electrical circuit is formed from a power source connected to the active and counter electrodes. The current flows from the active electrode, through the medicament pad, nail and the treatment site. From the treatment site the return current flows to the counter electrode and to the power source. Alternatively, the current may flow from the counter electrode to the active electrode. In addition, the toenail or fingernail may be soaked in salt-water or other conductive fluid prior to electrokinetic delivery of the medicament to reduce the electrical resistance of the nail during medicament delivery. Other techniques may be used to increase the conductivity of the toenail or other treatment site, such as applying a cream or other film to the nail before the applicator is placed on the nail.

An electrokinetic apparatus has been developed to apply medicament to a treatment site of a mammalian user, the apparatus comprising: a flux concentrator including an active electrode and a counter electrode adapted to be applied to the surface of a toenail or fingernail of the user, and a medicament matrix adjacent the active electrode and arranged to be sandwiched between the active electrode and nail.

An electrokinetic apparatus has been developed comprising: an applicator cartridge including a front surface adapted to be applied to a treatment site including at least one of a toenail, fingernail and skin surface of a user; an active electrode, a counter electrode electrically isolated from the active electrode, where the active electrode and counter electrode are mounted on the front surface of the cartridge, and a matrix carrying a medicament or a medicament and an electrically conductive carrier, wherein the matrix is mounted over the active matrix on the front surface; an electrical power source connectable to the active electrode and the counter electrode to apply electrical current through the active electrode, matrix, the treatment site, and the counter electrode.

A method has been developed to electrokinetically deliver a medicament to a treatment site in a mammalian user, the method comprising: applying a first surface of a medicament matrix to a nail of a toe or of the user; applying a first electrode to a second surface of the medicament matrix; positioning the first surface of the medicament matrix to the nail; positioning a second electrode on the nail; applying electrical current to an electrical current path extending through the active electrode, medicament matrix, at least partially through the nail and to the second electrode, and delivering medicament from the matrix into the nail by electrokinetic transporting the medicament along the current path.

### DESCRIPTION OF THE DRAWINGS

FIG. 1 is a perspective view of an exemplary electrokinetic delivery device including an applicator unit with a flux concentrator applied to a toenail and electrically coupled to a hand held power and control unit.

FIG. 2 is a perspective view of an applicator unit with a flux concentrator applied to a medicament matrix on a toenail.

FIG. 3 is a cross-sectional, side view of the flux concentrator, medicament matrix and toe shown in FIG. 2.

FIG. 4 is a front view of a first alternative flux concentrator.

FIG. 5 is a front view of a second alternative flux concentrator.

FIGS. 6 and 7 are schematic views of an applicator sheet that is applied to a toenail (in FIG. 6) and that may be optionally trimmed to fit the nail (FIG. 7), wherein the flux concentrator is arranged in an upper layer of the applicator sheet.

FIG. 8 is a cross-sectional view of the flux concentrator and plastic sheet shown in FIGS. 6 and 7.

FIG. 9 is a schematic view of an alternative applicator embodied with an untrimmed plastic sheet that is applied to a toenail, wherein the flux concentrator is arranged on an underside of the plastic sheet.

FIG. 10 is a cross-sectional view of the flux concentrator and plastic sheet shown in FIG. 9.

FIGS. 11 and 12 are a front view of a flux concentrator having a center counter electrode and a plurality of rings of active electrodes (wherein the concentric positions of the active and counter electrodes may be reversed in alternative embodiments of the concentrators shown in FIGS. 11 and 12).

FIG. 13 is a view of a toe being soaked in a salt water to infuse a conductive liquid in the nail before electrokinetic delivery of the medicament to the nail.

FIG. 14 is a schematic view of an applicator unit with an applicator sheet applied to a toenail and surrounding soft tissue wherein the flux concentrator is arranged on an underside of the plastic sheet with a counter-electrode applied to the bottom of the toe or elsewhere on the body.

FIG. 15 is a cross-sectional view of the flux concentrator shown in FIG. 14 illustrating flux pathways.

### DETAILED DESCRIPTION OF THE INVENTION

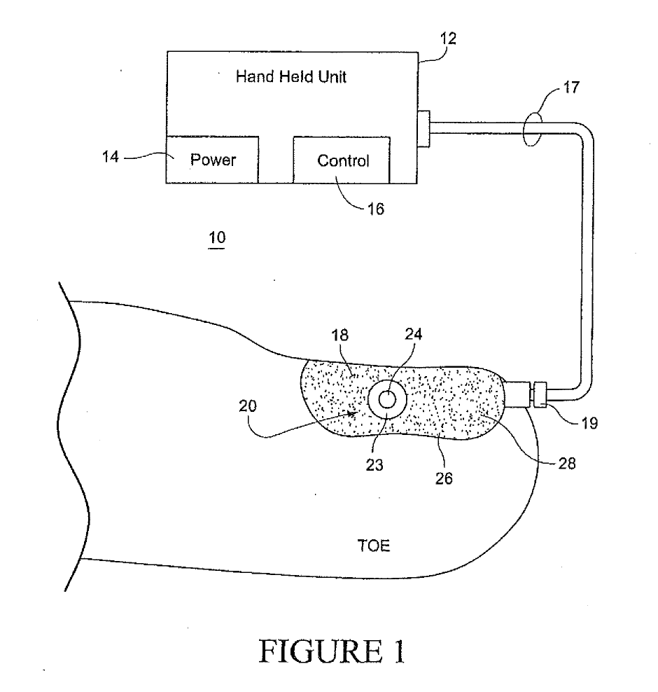

FIG. 1 is a schematic diagram of a portable, self contained, lightweight, compact, electrokinetic medicament delivery device or medicator 10 (collectively a “device”) for application to a treatment site on a fingernail or toenail of an individual (or a region of skin having a high resistance or particularly hard such as a wart or callous). The device 10 includes a hand held unit 12 housing a power source 14, e.g., batteries, and an electronic controller 15 with associated circuits for activating the device and applying electrical power to electrodes and a medicament matrix applied to the treatment site. Preferably, the electrical power source provides direct current (DC) to deliver medicament to a treatment site. However, in some applications alternating current (AC) or a combination of AC and DC may be applied to the medicament matrix and treatment site. The hand held unit 12 may include a grip for a user to grasp the unit, wherein the grip may be a finger grip or hand grip. The power source may be incorporated into the applicator sheet 18, such as by means of a thin film battery forming a layer of the applicator sheet.

A conductive cable(s) 17 provides electrical power and control coupling between the power and control circuits in the hand held unit 12 and a medicament applicator sheet 18 applied to a toenail 26. A connector 19 provides a releasable electrical coupling between the electrodes of a flux concentrator 20 included in the applicator sheet 18, e.g., laminated in the applicator sheet 18. The flux concentrator 20 provides active and counter electrodes to form a conductive electrical path that passes through the sheet 18, including a medicament layer of the sheet, and into the toenail 26 to which the sheet is applied.

The flux concentrator 20 is shown in a first embodiment in FIG. 1 in which an active electrode 24 is centrally positioned in the sheet 18 and on the toenail 26 and a relatively large counter electrode 28 (indicated by the shaded region of the applicator sheet 18) substantially covers the toenail. An annular isolation region 23 of the sheet 18 electrically separates the active and counter electrodes. The counter electrode(s) surrounds the annular isolation region 23 and the active electrode 24. Alternatively, the counter electrode may be arranged as the center of the applicator sheet and the active electrode may form a rim at the edge of the sheet with the isolation region separating the active and counter electrodes.

In the embodiment shown in FIG. 1, the active electrode 24 and hence the medicament matrix is confined to a center portion of the toenail and is distant from the soft skin tissue at the edges of the nail. The counter electrode 28 may be in direct electrical contact with the upper surface of the toenail 26. A medicament matrix (not visible in FIG. 1) is sandwiched between the active matrix sheet 28 and the toenail.

The electrokinectic medicament delivery device 10 drives, e.g., electrokinetically transports, medicament interposed between the active electrode of the flux concentrator 20 and the treatment site, e.g. toenail, upon completion of an electrical circuit through the device, the active electrode, the medicament or hydration material carrying the medicament (collectively referred to as “medicament”), the treatment site in the individual's body and the counter electrode 28, e.g., tactile or ground electrode. The treatment site may be a fingernail or toenail of a user, a callous, a wart or other hard skin surface on the user.

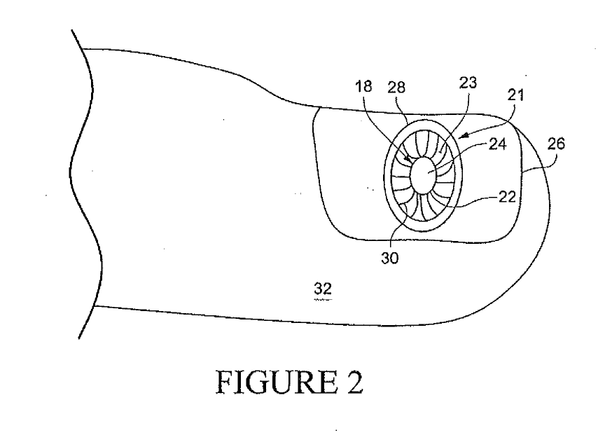

FIG. 2 is a perspective view of an applicator sheet with another embodiment of a flux concentrator 21. The active electrode 24 is centrally located on the toenail and the counter electrode 28 surrounds the active electrode. The active electrode may be relatively small as compared to the surface area of the toenail and is intended to be positioned on a central location of the toenail. However, the active electrode may cover a substantial portion of the toenail, such between three-quarters and one-half of the upper surface area of the toenail. Alternatively, the active electrode 24 may cover the entire nail and a portion of the soft skin tissue surrounding the nail. The soft skin tissue may be shield by a mask between this tissue and the active electrode. The counter electrode 28 may be arranged to surround the active electrode or be applied to the bottom of the toe or elsewhere on the body.

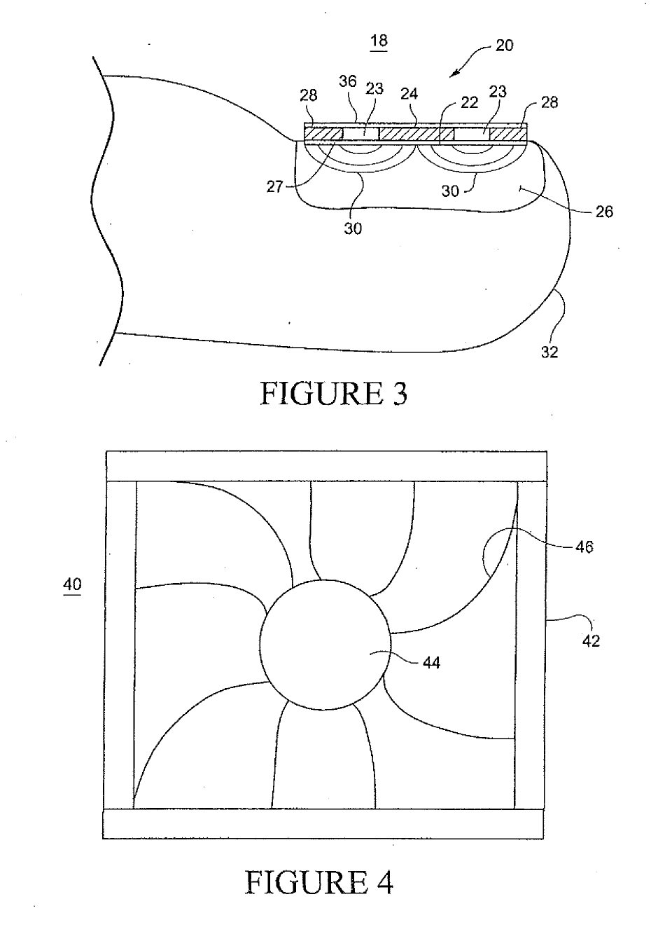

Current flows from the active electrode 24, through the medicament matrix 22 (which underlies the active electrode), the toenail 26 and possibly into the soft skin tissue below the nail if the treatment site is in the skin tissue. Current may flow through the nail into underlying soft tissue. The depth to which the current flows depends on the power applied and the distance between the active and counter electrodes, if both electrodes are in the same plane, such as on the nail as shown in FIG. 3.

The flux concentrator 20, 21 is particularly suited for application to toenails, fingernails and certain soft tissue skin surfaces, such as warts and calluses. High resistance nail and certain soft tissue skin surfaces requires a higher voltage to drive any current into the treatment site. The current density is depends on the area through which current is flowing into the treatment site. The benefit of the flux concentrator disclosed here is to effectively manage current to nail and skin areas that have differing resistance. The flux concentrator may manage current by protecting soft tissue from high voltage and inadvertent contact to an active electrode or reducing the need for a high voltage supply by covering high and low resistance tissues (nail and skin) simultaneously. They also may be used to concentrate flux to certain treatment sites such as toenails, warts and calluses, where higher concentrations of drug are desired, The flux concentrator and medicament matrix are designed and shaped to be applied to the surface of a toenail, such as a nail with a fungal invention. The flux concentrator and associated medicament matrix may also be applied to a wart or callus. For purposes of illustration, the active flux concentrator and medicament matrix described herein are intended for a toenail. However, they may also be adapted to a fingernail, hard skin surface, such as a wart or callous or other high resistance skin area.

The medicament may be used to treat, for example, onychomycosis, which is a fungal invasion of the nail. The fungal infection may be due to a dermatophyte, yeast, or nondermatophyte mould. The medicament is intended to destroy the fungus or at least cause the fungal invasion to subside.

To deliver medicament to the treatment site, the applicator sheet 18 delivers current through the active electrode 24, the medicament matrix 22 (between the active electrode and the toenail), the toenail 26 and to the treatment site in or under the toenail. The current causes medicament to be transported from the matrix to the treatment site due to electrokinetic effects, e.g., iontophoresis. The path of the current includes a return path from the treatment site, through the counter electrode 28 and to the power source 14 in the hand held unit.

The flux concentrator 20 focuses, confines and/or directs the current to the treatment site and, particularly, to a treatment site in or under a high resistance nail or other portion of the skin. The flux concentrator, preferably, is shaped to fit the toenail or hard skin surface. The flux concentrator 20 includes an active electrode 24 and a counter electrode 28. The flux concentrator may be included in one or more layers of the applicator sheet. Further, the medicament matrix 22 may be another layer of the application sheet and is in a layer below the active electrode such that the matrix is sandwiched between the active electrode and the toenail. The active electrode may be, for example, a conductive layer, a conductive mesh, array of other network of conductive wires or a conductive panel superimposed over and/or applied to an upper surface of the medicament matrix.

The flux concentrator 20 may be configured to apply a relatively high current density to the toenail 26 and, particularly, the nail region proximate to a treatment site in or under the toenail. The current path, e.g., electromagnetic flux, is intended to extend into the nail 26 and possibly to the soft skin tissue of the toe 32 underlying the toenail 26. Further, the flux concentrator may substantially reduce the current density flowing to soft skin tissue near the nail. The reduction in current density as current flows from the small active electrode to the larger counter electrode is illustrated by the radial flux lines 30 extend outward from the center active electrode 24 and towards the outer counter electrode 28. As the radial flux lines extend radially outward, there is a corresponding reduction in current density.

In general, the active electrode 24 is centrally located in the flux concentrator 20 and the counter electrode 28 is arranged outward of the active electrode, such as at the periphery of the flux concentrator. The flux concentrator includes a dielectric gap region 23 (also referred to as an annular isolation region) between the active and counter electrodes. The gap 23 between the active electrode and counter electrode should be sufficiently large to reduce or eliminate current, e.g., eddy currents, on the upper surface of the toenail.

The dielectric region 23 may be an air gap between the active electrode and counter electrode. The air gap may be an air chamber formed between the outer edges of the active electrode and medicament layer and the inner edges of the counter electrode. The upper surface of the air chamber is defined by an upper layer of the applicator sheet and the lower surface is defined by the toenail or medicament matrix. Optionally, a non-conductive material, e.g., a cellulosic fiber mesh or solid plastic material, may be inserted in the chamber to prevent collapse of the chamber when the applicator sheet is applied to the nail. If a solid material is in the chamber, the chamber is no longer an air gap but is a chamber filled with a dielectric material to prevent current between the active and counter electrode that does not pass through the medicament matrix and toenail.

FIG. 3 shows, in cross-section and side view, a multilayered applicator sheet 18 having an upper substrate layer 36, a middle layer including the flux concentrator 20 and lower layer including a medicament matrix 22. The applicator sheet may include a bottom layer 27 that is an adhesive coating to adhere to the toenail. The applicator sheet may be stored in a container, such as an envelope, that is opened shortly before the applicator sheet is to be applied to a toenail.

The active electrode 24 and counter electrode 28 may be composed of, for example, metal, a metallized polymer or a conductive polymer such as polyaniline, polypyrrole, or a polymer rendered conductive by means of a conductive dopant. The flux concentrator may include a planar substrate 36, e.g., a flexible dielectric or low-conductivity plastic material. The planar substrate provides support for the electrodes. The material for the planar substrate may also fill the chamber of the isolation region 23 and provide the dielectric material between the electrodes.

The active and counter electrodes are mounted to an underside of the substrate 36 or are embedded in the substrate. Electrical wires or other conductive pathways provide electrical connections between the active and counter electrodes and a connection 19 (FIG. 1) to the power supply 17 and control electronics in the hand held unit 10. The front side of the flux concentrator with the electrodes 28, 24 is preferably applied to a backside of the medicament matrix.

The medicament matrix 22 may be confined to the area under the active electrode. The medicament matrix may include a matrix carrier supporting the medicament. The shape and surface area of the medicament matrix may be substantially the same as that of the active matrix. Acceptable materials for the matrix carrier include but are not limited to variable loft nonwoven and woven materials such as melt-blown, needlepunched, spunbonded, spunlaced or other processed natural fibers, polyolefin, polyester, rayon, nylon, and blends of these, reticulated polyether and polyester polyurethane foams, and silicone foams. Low void volume materials may also be used such as crosslinked hydrogels, interpenetrating polymer networks, scaffolds for immobilizing the active prior to iontophoretic release, highly viscosified formulations, and other matrices that do not rely upon a delivery from a liquid formulation. The matrix carrier may also contain functional components such as reinforcing scrims, networks, and other support structures to facilitate manufacture of the finished product. These layers may also be conductive to ensure homogeneous electrical contact with the drug formulation contained in the matrix. Additionally, the matrix may contain one or more layers carrying arrays of microneedles or other surface features designed to physically penetrate some or a portion of the toenail to promote delivery of medicaments to or through the nail.

The medicament is supported by a matrix carrier of the medicament matrix 22. The medicament or the medicament or hydration material carrying the medicament (collectively referred to as “medicament”) are contained in the matrix carrier. The matrix carrier may be a thin flexible pad. The carrier may be tailored, e.g., cut, by the user to conform to the toenail to which the medicament matrix is to be applied.

The flux concentrator 20, 21 is generally applied to a first surface of the matrix carrier. An opposite surface of the matrix carrier is applied to the toenail. The opposite side of the matrix carrier may include an adhesive to secure the carrier to the toenail. Alternatively, the medicament matrix 22 may be mounted to a front surface of a cartridge head, either under a factory seal lid on the cartridge or after being tailored to fit the toenail.

The current may flow from the active electrode through medicament matrix in a direction at least partially perpendicular to the plane of the matrix, through the upper surface of the toenail (in a direction generally perpendicular to the plane of the nail) and to the treatment site. The current density is relatively high in the medicament matrix and toenail immediately below the active electrode. The high current density causes medicament to be transported from the matrix into the toenail and, in some embodiments, through the nail and to the soft skin tissue underlying the nail.

The current path defined by the radial flux lines 30 extends from the active electrode, through the medicament matrix, into the toenail and to the counter electrode. The current path spreads out from the active electrode as it flows to the counter electrode. By spreading the current path, the current density in the path become progressively reduced as the effective area or volume of the current path increases. Preferably, the current density reduces to levels that do not irritate, harm or burn the soft tissue below and adjacent the toenail. In addition to reducing current density in soft tissue, the flux concentrator may also concentrate drug delivery to desired treatment sites, e.g. more in the nail and less in soft tissue or the other way around as desired.

The counter electrode 28 provides an effective boundary to the current path between the active electrode and counter electrode. Arranging the counter electrode around the active electrode maintains the current path through the user with the perimeter of the counter electrode. No substantial amount of current should flow beyond the perimeter of the counter electrode from the active electrode. By arranging the counter electrode such that it fits entirely on the toenail, the current from the active electrode should not appreciably flow into the soft skin tissue of the toe that surrounds the toenail.

In the flux concentrator embodiment shown in FIGS. 2 and 3, the counter electrode 28 extends entirely around the active electrode 24. The counter electrode is positioned sufficiently far from the active electrode to ensure that the current path passes through the treatment site in the toenail and/or in the soft tissue below the toenail. If the counter electrode is close to the active electrode, a strong dielectric material in the isolation gap 23 between the electrodes may be necessary to prevent substantial current between the electrodes that does not reach the treatment site.

The active electrode 24 of the flux concentrator 20 and possibly the entire flux concentrator may have a smaller surface area than the medicament matrix. If medication or the medicament pad contacts the skin, the concentrator directs all (or substantially all) current paths to the high impedance nail on which the concentrator is applied. If the medicament matrix is trimmed to avoid the skin, there could still be a small contact area(s) between the matrix and soft skin tissue. These small contact areas may result in localized concentrations of current. Leaving the medicated contact area large and allowing significant overlap between the medicament matrix and soft tissue should avoid small contact areas between the matrix and soft tissue. Current that may flow through the matrix (despite the flux concentrator) to the skin will be distributed over the large contact area

FIG. 4 is a schematic diagram showing a front view of a second embodiment of a flux concentrator 40. The flux concentrator and medicament matrix may be mounted to an applicator sheet or other portable cartridge head of an electrokinetic medicament delivery device.

The counter electrode 42 forms a conductive rail or border around the perimeter of the active electrode 44. For example, the counter electrode 42 may be formed of straight conductive strips arranged in a square (or other trapezoid) to form a conductive rail around the active electrode. The counter electrode 42 may be shaped to conform to the surface of the toenail to which it will be applied. The current density become progressively less intense (see flux lines 46), as current flows away from the active electrode 44, through the medicament matrix and treatment site and to the counter electrode 42.

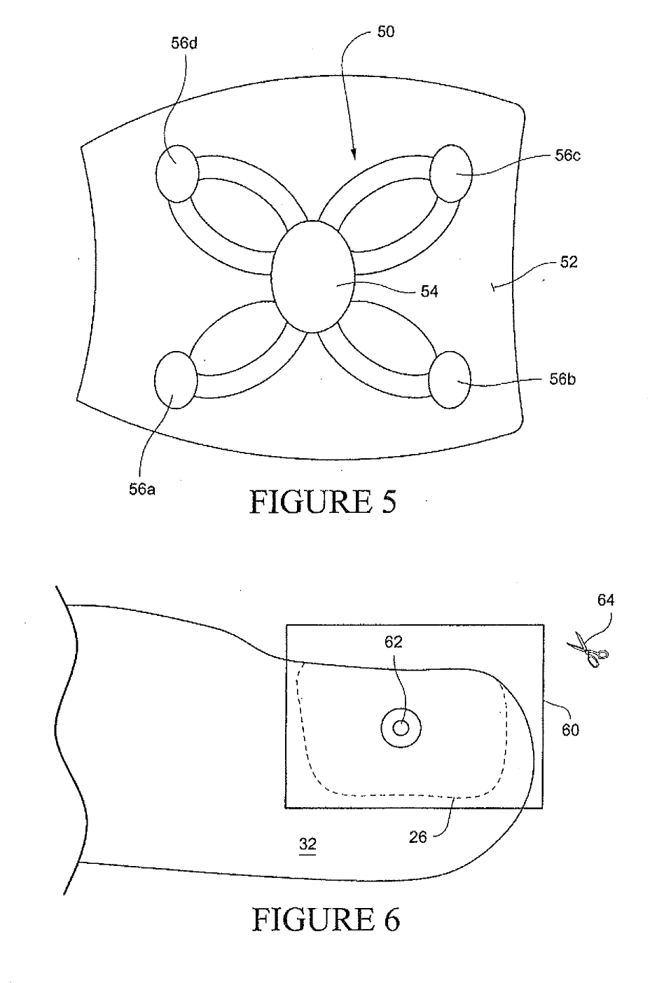

FIG. 5 is a third embodiment of a flux concentrator 50 on a toenail 52. The flux concentrator may be laminated in an applicator sheet, arranged in a a cartridge head of an applicator device or otherwise arranged with a medicament matrix to be applied to a toenail. The active electrode is preferably superimposed with the medicament matrix, where the matrix is sandwiched between the active electrode and toenail. The active electrode 54 is in a center region of the flux concentrator. An array of individual counter electrodes 56 (56a, 56b, 56c and 56d) are arranged around the perimeter of the active electrode 54. The array of counter electrodes forms a discontinuous border fence around the active electrode. Current from the active electrode flows to one or more of the counter electrodes 56. The counter electrodes 56 may be arranged such that they abut the toenail 52.

A controller 16 (FIG. 1) in the electronics of the hand held unit may independently control the individual counter electrodes 56 a-d and determine when and whether each counter electrode conducts current. For example, the controller may regulate a switch in the conductive line from the individual counter electrode to the power supply or to a central connection for the current return to the power supply. Independent control of the counter electrodes, provides some control over the current flow, flux pattern and may be use to prevent excessive current flow through any one of the counter electrodes. For example, if the controller senses an abnormally high current flow, the counter electrode having the high current may be switched off. An abnormally high current flow may be a 50 to 10 percent greater current through one counter electrode as compared to the average of all counter electrodes.

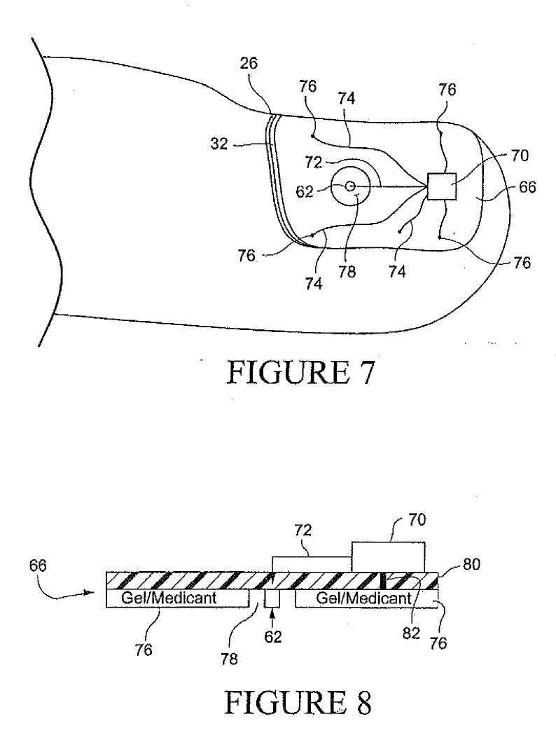

FIGS. 6 and 7 are a schematic view of an applicator embodied as sheet that is applied to a toenail and trimmed to fit the nail, wherein the flux concentrator is arranged on an underside of the plastic sheet. The applicator sheet includes a drug containment hydrogel and/or foam layer 60 and a counter-electrode, 62 formed from a continuous sheet. Electrodes 76 are deposited on the top surface of the sheet with an insulating ring 78 between the counter electrode 62 and the remainder of the sheet 60 which has the electrical contacts 76. The sheet may be either trimmed to conform to nail surface (as shown in FIG. 1) or it may be used without trimming. If the sheet is not trimmed, current may flow into the nail and through surrounding soft tissue back to the counter electrode. An insulating mask layer may be applied to the soft skin tissue before the untrimmed applicator sheet is applied to the toenail. FIG. 3 shows current flow and the cross-sectional construction of a version of this applicator that is applied to the nail only. Similar construction and patterns will exist if the sheet is applied over soft tissue in addition to the nail. The flux concentrator may include conductive traces on its top surface. Medication and/or counter-electrode conductive material is under the conductive traces, the tissue into which current is delivered lies beneath the medication layer (except for the alternative design where the counter electrode is located under the toe or elsewhere on the body.)

As shown in FIG. 6, a rectangular applicator sheet 60 is uncut and is substantially larger than the toenail 26. The uncut sheet applicator 60 is placed on the nail 26 such that the counter electrode 62 is preferably centered on the nail. An adhesive on an exposed surface beneath the sheet may adhere to the nail. The uncut sheet is trimmed by scissors 64, a knife, another cutting device or by tearing the sheet. The trimmed sheet applicator 66 (FIG. 7) overlies substantially the entire exposed surface of the toenail 26. The trimming operation may leave a narrow rim 68 of the nail between the perimeter of the trimmed sheet 66 and the soft skin adjacent the nail. Trimming the sheet to confine the sheet to the nail provides one means to prevent excessive currents in the soft tissue adjacent the nail.

Once trimmed the applicator sheet is applied to the toenail. A connector 70 on the sheet may be attached to a conductive cable which provides a path for electrical power and control signals between the sheet and a control device (such as the hand held united 12 in FIG. 1). The connector 70 includes a first electrical path 72 to the counter electrode 62 and a second electrical path 74 to one or more active electrodes 76. The active electrode(s) may be a ring electrode or an array metallic electrodes embedded in the sheet applicator 66. In addition or alternatively, the active electrode may be a conductive gel on a lower surface of the sheet and sandwiched between the sheet and nail. The second electrical path may be a plurality of paths as shown in FIG. 7, a single path to a ring active electrode or a network of paths to one or more active electrodes. The first electrical path 72 is separate from the second path 74. An isolation dielectric ring 78 in the sheet 66 electrically separates the counter electrode 62 from the active electrodes 76. While FIGS. 6 and 7 show the counter electrode in the center of the applicator sheet and toenail, the counter electrode may be arranged off-center.

FIG. 8 is a cross-sectional view of the plastic sheet applicator 66 shown in FIGS. 6 and 7. The sheet may include a plastic carrier layer 80 and underlying gel layers that are sandwiched between the plastic carrier 80 and the toenail. The gel layers are preferably conductive so that they may serve as the active electrode 76 and as the counter electrode 62. If the active electrode is formed by a conductive gel layer the gel may serve as the active electrode 76.

The counter electrode 62 and active electrode 76 may be formed of a conductive gel, such as the gels used for attaching electrocardiogram (ECG) electrodes to the skin of patient. The gel of the active electrode includes the medicament to be electrokinetically applied to the treatment site in the nail for example. The gel for the counter electrode need not have medicament. Conductive gels suitable for the active and counter electrodes are commonly available, safe for topical use on the skin, and may be formed of a composition including water, aluminum oxide, propanediol, sodium polyacrylate, methylparaben, and propylparaben. An adhesive may be included in the gel to secure the plastic carrier layer and connector 70 to the nail. The active electrode and conductive electrodes may be formed of a substantially solid gel that retains the desired shape of the electrodes as the sheet applicator is applied to the nail, trimmed and connected to an electrokinetic device. The solid gel electrodes may deform to conform to the surface of the toenail.

A conductive lead or contact 82 may extend from the connector 70, through the sheet 80 and to conductive gel forming the active electrode 76. A conductive path 72 may extend over the exposed surface of the sheet 80, through the sheet and to the gel forming the counter electrode 62.

A non-conductive annular ring 78, e.g., a plastic ring, a paper ring or an annular dead air space, attached to the underside of the sheet 80 separates the counter electrode gel from the active electrode gel. The non-conductive annular ring 78 has no medicament and should be sufficient to electrically isolate the active and counter electrodes. The non-conductive annular ring 78 should provide electrical isolation for a voltage difference of, for example, 200 volts between the active and counter electrodes.

The applicator sheet 60 or 66 may be formed of a flexible biocompatible plastic sheet. Applied to this sheet is a solid gel electrode with a layer of carrier film that is non-conductive. The counter-electrode could be formed by die-cutting the isolation region and cutting through the conductor layer for interconnect.

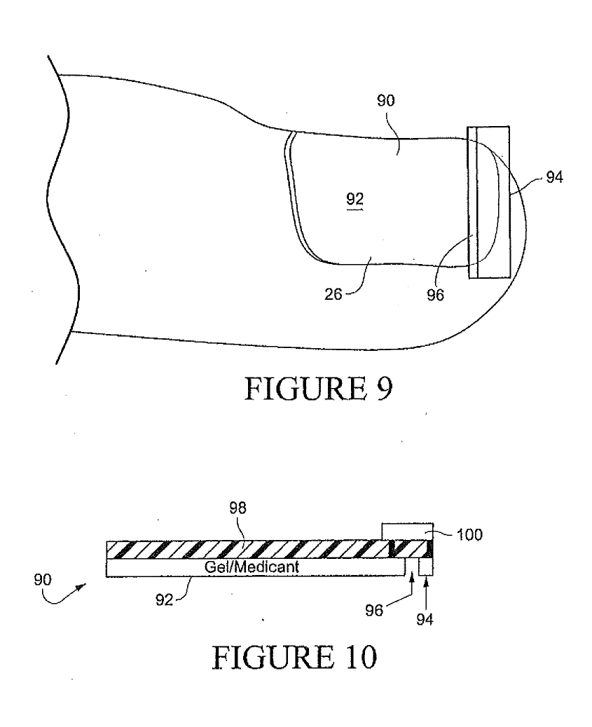

FIGS. 9 and 10 show an alternative applicator sheet 90 applied to a toenail 26. The applicator sheet has a solid gel active electrode 92 that covers substantially the entire surface of the nail except for a surface near the front edge of the nail. The counter-electrode 94 is arranged at the front edge of the sheet 90 and is formed as a conductive gel that is in contact with the front edge of the toenail. The counter electrode is separated from the remainder portion of the rectangular shaped sheet with the active electrode gel film by an isolation wall 96 that may be formed by a die-cut in the conductive gel that forms the active and conductive electrodes.

FIG. 9 shows an electrokinetic medicament applicator sheet 90 including a top, conductive layer 98 and a second gel layer 92 containing medicament (drug doped hyrdogel or drug filled foam) with an insulating segment 96 and a counter electrode area 94.

Current flows from the medicament layer 92, through the nail and underlying soft tissue back to the counter electrode 94. The counter electrode is in contact with the tip of the toenail or other portion of the body of the users. For example, the counter electrode may be folded to be applied to the tip or bottom on the toe. If applied to the tip or bottom of the toe, the counter electrode may be a standard electrocardiogram (ECG) electrode. Current will flow primarily through soft tissue, but also through nail, delivering drug to both nail and surrounding soft tissue. Applying the counter electrode to a soft skin area reduces the amount of high resistance nail through which current must pass and thereby reduces the overall voltage level required to deliver drug to the treatment site.

An untrimmed portion of the sheet over the font edge of the nail is shown in FIG. 9. The untrimmed portion corresponds to a counter electrode rectangular strip 94 that includes a solid gel counter electrode that is applied to the nail. The counter electrode strip 94 is trimmed to conform to the surface of the nail near the front edge of the nail. The counter electrode establishes an electrical connection with the nail at the font edge of the nail. A non-conductive strip 96, e.g. an air chamber or non-conductive plastic strip, is between the counter electrode gel strip 94 and the active gel electrode 92. Although not shown in FIG. 9, a connector is provided on the applicator sheet 90 to couple the active electrode and counter electrode to an electrokinetic device having a power supply and controller.

FIG. 10 shows the applicator sheet 90 in cross-section to show the solid gel active electrode 94, solid gel strip counter electrode 92 and the isolation strip 96 separating the two electrodes. A plastic sheet 98 provides a substrate supporting the gel electrodes and isolation region. The non-conductive strip 96 may be formed by forming a die cut through a layer of conductive gel on the sheet. The die cut forms the active electrode and the counter electrode by removing the gel from the isolation strip 96.

A connector 100 provides a pair of electrical connections between a twin wire cable (not shown) and the electrodes, such that a conductive path is formed through the connector to the active electrode, the nail, counter electrode and back through the conductor and to the cable.

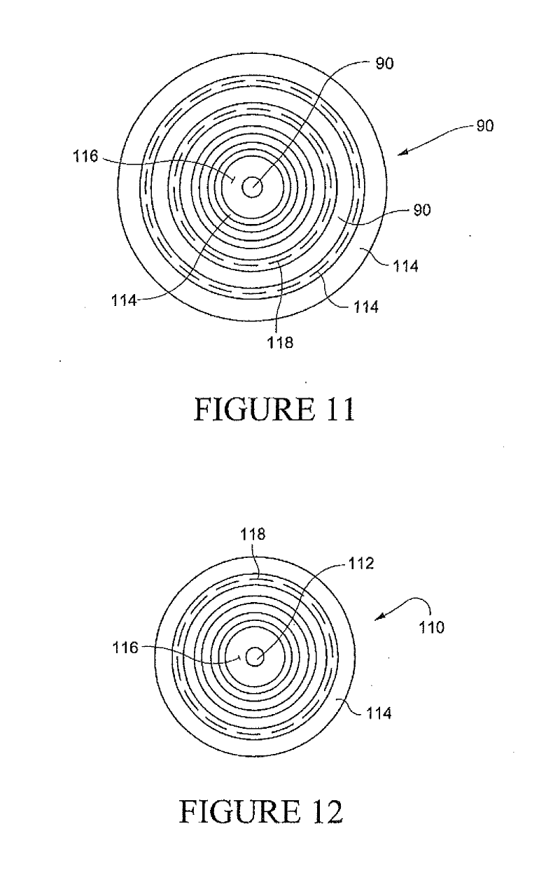

FIGS. 11 and 12 are a front view of a flux concentrator 110 having a center counter electrode 112 and a plurality of rings of active electrodes 114. The flux concentrator 110 may have a similar construction as the sheet applicators shown in FIGS. 6 to 10. In particular, a plastic sheet may provide a substrate for the active and counter electrodes which are formed by solid conductive gels applied to a surface of the plastic sheet. An isolation ring 116 electrically isolates the counter electrode from the surrounding active electrodes. Die cuts 118, e.g., perforated lines, may be in the substrate sheet and cut in the annular area between the active electrodes. The die cuts allow a user to easily remove and discard an outer active electrode from the flux concentrator 110. By removing one or more of the outer active electrode rings, the diameter of the concentrator 110 can be reduced. The reduction in diameter allows the user to size the concentrator 110 to the toenail, skin area or other treatment site to which the concentrator is to be applied. Reducing the diameter of the concentrator also provides a means to avoid having current flow to soft tissue surrounding a toenail on which the disc concentrator 110 is applied.

An alternative flux concentrator includes an active electrode in the center, and counter electrodes in concentric rings around the active electrode. Concentric rings of counter electrodes provides a flux concentrator that may be easily tailored to fit a specific toenail.

The flux concentrators disclosed herein direct current to a fingernail, toenail or other high impedance skin region and prevent high current to surrounding soft skin tissue. The concentrator may be used to direct high, limiting current densities to the high impedance toenail or finger nail and avoid high current densities at the soft skin tissue.

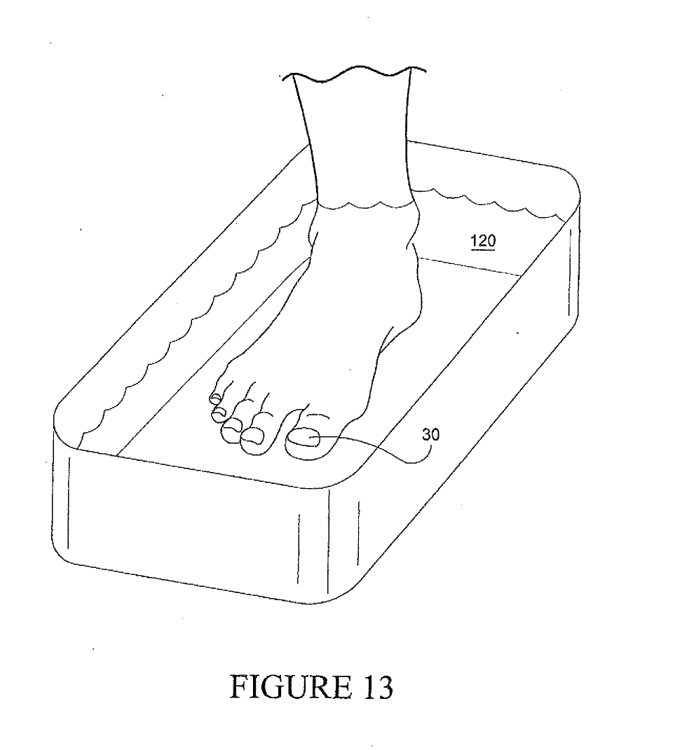

FIG. 13 is a view of a toe 30 being soaked in a salt water bath 120 to infuse a conductive liquid in the nail before electrokinetic delivery of the medicament to the nail. Several of the applicator embodiments disclosed herein are suitable for applying high voltages needed to deliver medicament to a toenail. Iontophoretic drug delivery to a toenail generally requires a relatively high voltage potential, e.g., greater than 100 volts, to apply sufficient current, e.g., greater than 100 μA, to deliver medicament into the nail. A supplemental technique is to reduce the resistance of the toenail to lower amount of voltage needed to deliver medicament to a toenail. Lowering the resistance of the toenail may be accomplished by applying soaking the toe in a solution to infuse conductive elements into the nail, applying a conductive cream or film to the nail or otherwise infusing conductive elements into the nail. Reducing the resistance of the nail, provides better electrokinetic delivery of medicament. Specifically, infusing salt water in the pores of the nail allows the salt water to conduct electricity through the nail and thereby allow for a lower applied voltage which reduces the risk of high voltages being applied to the skin and the costs associated with applying high voltages.

Soaking a toenail allows salt water to infuse into the pores of the nail. The salt water (also referred to as a saline solution) saturates the nail with sodium and chlorine ions that can reduce the electrical resistance of the nail. Other ionic solutions may be used to soak the toenail such as MgCl₂; a solution including the active formulation of the medicament, e.g., a ionized form of the active ingredient of the medicament, to be infused electrokinetically; and other components such as penetration enhancers, e.g., benzoic acid (0.1 to 0.2% w/w (by weight)), salicylic acid (1.0 to 5.0% w/w and/or fatty acids. Depending on the formulation of the composition applied to the nail and how quickly the formulation may swell the nail with conductive compositions, the formulation may be applied by saturated wipes or as a film or cream—rather than by soaking the toe. In addition the formulation may applied under pressure, such as by a syringe applicator, or a ballistic delivery mechanism to infuse the formulation into the nail before the electrokinetic applicator is placed on the nail.

The toenail may be soaked by placing the toe in a saltwater bath for a period of 10 minutes to 30 minutes, for example. Infusing salt water into the nail reduces the resistance of the nail.

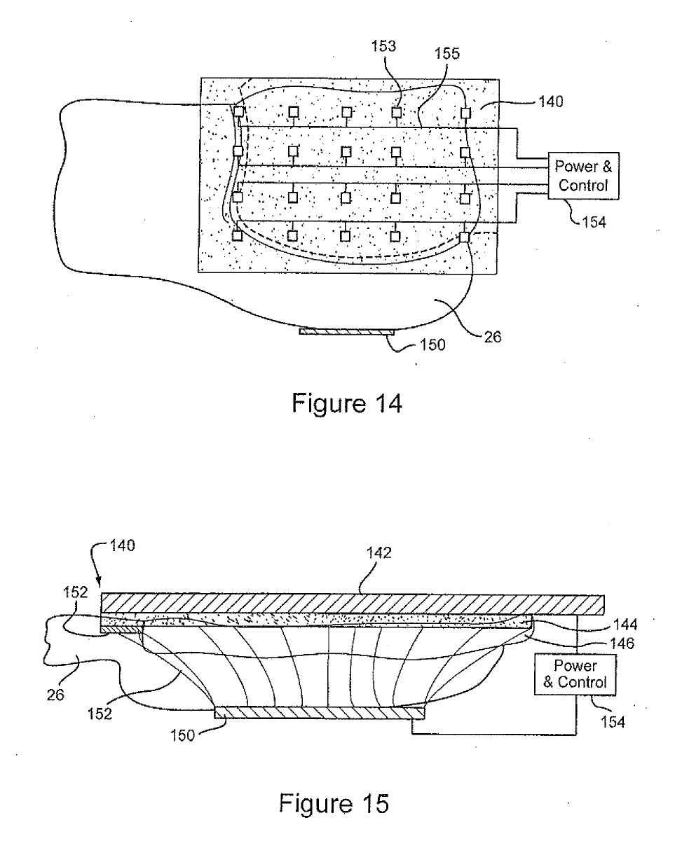

FIGS. 14 and 15 show an alternative applicator sheet 140 applied to a toenail 26. The applicator sheet 140 may also be configured to be applied to a wide area surface of the skin.

The applicator sheet 140 has a solid gel active electrode 142 that covers the entire surface of the nail and a portion of the soft tissue surrounding the nail. A counter-electrode 150 is applied to the bottom of the toe 26 and is preferably formed as a conductive gel that is in contact with the bottom of the toe. It is not necessary for the applicator sheet 140 to be trimmed to cover only the toenail. However, the sheet may be trimmed as indicated by the dotted line in FIG. 14.

FIG. 15 shows a cross-sectional view of the applicator sheet 140, counter-electrode 150 and resulting lines of flux 152 as current flows from the active electrode 142, through the medicament layer 144, through the nail 146 and surrounding tissue to the counter electrode.

The electrode pattern of the gel active electrode 142 arranged on top of the medicament layer 144, may be individually controlled electrical contacts 153. Electrical lines 155 extend through the gel layer of the active electrode between each electrical contact 153 and a power source and controller 154. Current through each contact 153 may be controlled by the electronic controller 154, such as by selectively turning on or off individual contacts, and limiting the application of current to one or more contacts to enhance flux control through the hard nail, soft tissue or both. The controller may sense the impedance through each contact to determine whether to limit current or turn on or off the contact.

As an alternative or in addition to regulating current by monitoring the impedance of the contacts, a mask 152 may be applied to the soft tissue areas of the toe before the applicator sheet 140 is applied to the toenail. The applicator sheet may overlie the mask 152 such the applicator sheet is not in direct contact with the soft tissue. The mask may be an insulating sheet, such as a non-conductive plastic sheet, to prevent current flow through the mask. The mask has an opening that allows contact of the medicament layer 144 with the nail surface. The mask has adhesive and electrical isolation properties that prevent leakage of medicament and current from reaching soft tissue. The mask may cover the soft tissue on the outside of the dotted line shown in FIG. 14.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1-41. (canceled)

42. A method of electrokinetically delivering a medicament to a treatment site of a subject, comprising:
soaking the treatment site in salt water;
applying to the surface of the treatment site a flux concentrator including an active electrode and a counter electrode in a sheet;
applying a medicament matrix between the active electrode of the flux concentrator and the treatment site, wherein the medicament matrix holds a medicament; and
applying an electrical current through the active electrode, matrix, treatment site, and counter electrode to deliver the medicament to the treatment site.

43. The method of claim 42, wherein soaking the treatment site in salt water infuses a conductive solution into the treatment site.

44. The method of claim 42, wherein soaking the treatment site in salt water reduces the electrical resistance of at least a portion of the treatment site.

45. The method of claim 42, wherein the treatment site comprises at least a portion of the subject's nail.

46. The method of claim 42, wherein the salt water comprises NaCl.

47. The method of claim 42, wherein the salt water comprises MgCl₂.

48. The method of claim 42, wherein the treatment site is soaked in salt water between about 10-30 minutes.

49. A method of electrokinetically delivering a medicament to the nail of a subject, comprising:
reducing the electrical resistance of at least a portion of the nail surface;
applying to the surface of the nail an active electrode;
applying a medicament matrix between the active electrode and the nail, wherein the . medicament matrix holds a medicament;
applying a counter electrode to a surface of the subject; and
applying an electrical current through the active electrode, matrix, nail, and counter electrode to deliver the medicament to the nail.

50. The method of claim 49, wherein reducing the electrical current comprises infusing a conductive composition into the nail.

51. The method of claim 50, wherein infusing a conductive composition comprises soaking the nail in a saline solution.

52. The method of claim 51, wherein the saline solution comprises NaCl.

53. The method of claim 51, wherein the saline solution comprises MgCl₂.

54. The method of claim 51, wherein the nail is soaked in saline solution between about 10-30 minutes.

55. The method of claim 50, wherein the conductive composition is applied to the nail via a saturated wipe.

56. The method of claim 50, wherein the conductive composition is applied to the nail as a film or cream.

57. A method of electrokinetically delivering a medicament to the nail of a subject, comprising:
infusing a conductive composition into at least a portion of the nail to reduce the electrical resistance of at least a portion of the nail surface;
applying to the surface of the nail an active electrode and a counter electrode in a sheet;
applying a medicament matrix between the active electrode and the nail, wherein the medicament matrix holds a medicament; and
applying a reduced electrical current through the active electrode, matrix, treatment site, and counter electrode to deliver the medicament to the nail.

58. The method of claim 57, wherein the reduced electrical current is less than 100 μA.

59. The method of claim 58, wherein the conductive composition is a saline solution.

60. The method of claim 59, wherein the saline solution comprises NaCl.

61. The method of claim 59, wherein the saline solution comprises MgCl₂.
